(19) United States
(12) Patent Application Publication
(10) Pub. No.: US 2023/0313303 A1
(43) Pub. Date: Oct. 5, 2023

(54) COMPOSITIONS AND METHODS FOR REDUCING CELL-FREE DNA

(71) Applicant: The Medical College of Wisconsin, Inc., Milwaukee, WI (US)

(72) Inventors: Michael Mitchell, Elm Grove, WI (US); Aoy Tomita Mitchell, Elm Grove, WI (US)

(21) Appl. No.: 18/057,494

(22) Filed: Nov. 21, 2022

Related U.S. Application Data
(63) Continuation of application No. PCT/US2021/033453, filed on May 20, 2021.
(60) Provisional application No. 63/027,389, filed on May 20, 2020.

(51) Int. Cl. C12Q 1/6883; A61M 1/34; C12Q 1/6851

(52) U.S. Cl. CPC C12Q 1/6883; A61M 1/34; C12Q 1/6851; C12Q 1/6806

(57) ABSTRACT

This invention relates to compositions and methods for treating subjects in need of mechanical support, such as those who have had surgery and/or are in need of pro-inflammatory reduction. The mechanical support can comprise a filter that reduces the amount of cell-free DNA in the subject, in some embodiments. The subject may be treated with cf-DNA inhibitors in other embodiments.

### RELATED APPLICATION

This application claims the benefit of priority under 35 U.S.C. § 119(e) to U.S. Provisional Application No. 63/027,389, filed May 20, 2020, the entire contents of which are incorporated herein by reference.

### FIELD OF THE INVENTION

This invention relates to compositions and methods for reducing cell-free DNA in subjects in need of mechanical support, such as those in need of pro-inflammatory reduction.

### SUMMARY OF INVENTION

The present disclosure is based, at least in part, on the surprising discovery that subjects on mechanical support, such as a ventilator, extracorporeal membrane oxygenation (ECMO) device, or cardiopulmonary bypass device, can benefit from reduction of cell-free DNA, such as part of their mechanical support. Such subjects can be any subject undergoing cardiopulmonary bypass or mechanical support for any reason including, but not limited to, open heart surgery or support on ECMO. Such subjects can be any with an undesirable pro-inflammatory response or one at risk thereof.

In one embodiment, the mechanical support can include a filter that removes cell-free DNA from circulation. The mechanical support can comprise a ventilator and/or extracorporeal membrane oxygenation (ECMO) device and/or cardiopulmonary bypass device in any one of the compositions or methods provided herein. The filter can be CYTOSORB in any one of the compositions or methods provide herein.

In one embodiment, any one of the subjects provided herein can be treated with an agent that inhibits or reduces cell free-DNA (cf-DNA).

In one embodiment, the methods comprise steps of assessing the levels of total cf-DNA in the subject. These steps can be used to monitor the subject over time to assess the efficacy of the treatment and/or to identify those in need of or who would benefit from the treatment.

Provided herein are methods, compositions and kits related to such treatments. The methods, compositions, or kits can be any one of the methods, compositions, or kits, respectively, provided herein, including any one of those of the Examples or Figures.

In one embodiment of any one of the methods provided, the method further comprises obtaining a sample from the subject.

In one embodiment, any one of the embodiments for the methods provided herein can be an embodiment for any one of the compositions or kits provided. In one embodiment, any one of the embodiments for the compositions or kits provided herein can be an embodiment for any one of the methods provided herein.

In one aspect, any one of the methods provided herein is provided. In one embodiment of any one of the methods provided herein, the amount indicative of severity and/or risk of a complication is any one of the thresholds as described herein. In one embodiment of any one of the methods provided herein, the time for obtaining the sample is any one of the times described herein. In one embodiment of any one of the methods provided herein, the subject is any one of the subjects described herein.

In one embodiment of any one of the methods of treating, the treatment is for any one of the conditions provided herein. Examples of which are provided herein or otherwise known to those of ordinary skill in the art.

In any one of the methods provided herein the methods may comprise treating, determining a treatment regimen for, or providing information about a treatment to any one of the subjects provided herein.

### BRIEF DESCRIPTION OF FIGURES

The accompanying figures are not intended to be drawn to scale. The figures are illustrative only and are not required for enablement of the disclosure.

FIG. 1 illustrates an example of a computer system with which some embodiments may operate.

FIG. 2 includes two graphs and a table showing the correlation between total cell-free DNA and death, using a cutoff value of 50 ng/mL.

FIG. 3 is a graph using receiver operator characteristic (ROC) analysis on repeated measures using correlation to examine the relationship between death and total cf-DNA (whole blood and plasma). 1150 samples from 197 patients followed for at least one year following heart transplant were analyzed.

FIG. 4 includes two graphs and a table showing the correlation between total cf-DNA and death in pediatric samples (whole blood and plasma) following heart transplant.

FIG. 5 includes two graphs and a table showing the correlation between total cf-DNA and death in adult samples (whole blood and plasma) following heart transplant.

FIGS. 6A-6D show different experimental cutpoints (thresholds) for total cf-DNA and time to death. 50 ng/mL (FIG. 6A), 25 ng/mL (FIG. 6B), and 10 ng/mL (FIG. 6C) were examined. The results are tabulated in FIG. 6D.

FIG. 7 shows product-limit survival estimates for subjects based on total cf-DNA. The samples were taken from patients after heart transplant, and the time from the test to the events of death, cardiac arrest, or need for mechanical circulatory support, was examined.

FIG. 8 includes two graphs and a table showing the correlation between total cf-DNA and an event (death, cardiac arrest, or need for mechanical circulatory support).

FIG. 9 shows an analysis of three different cutoffs (thresholds): 50 ng/mL, 25 ng/mL, and 10 ng/mL.

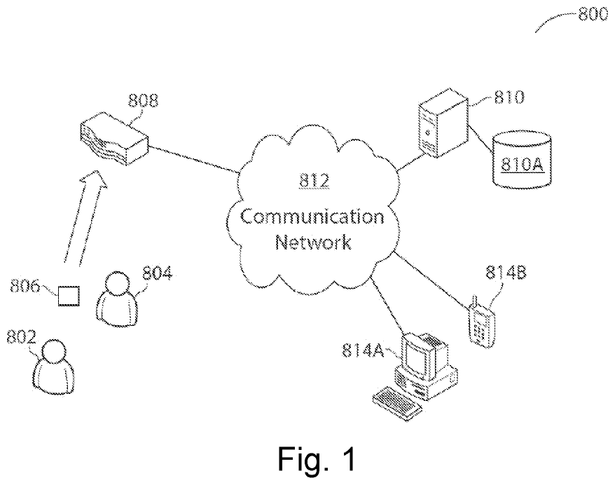

### DETAILED DESCRIPTION OF THE INVENTION

With its pro-inflammatory properties, levels in response to tissue injury, and other commonalities, it is believed that reduction of cell-free DNA can be beneficial in subjects on mechanical support, such as ECMO or cardiopulmonary bypass. Thus, provided herein, are methods of treating subjects on or in need of mechanical support.

Such treating can include with a device that includes a filter to reduce the amount of cell-free DNA in the subject. In one embodiment, the device includes an extracorporeal filter, such as an extracorporeal cytokine adsorber (e.g., CytoSorb®). In one embodiment, the treatment comprises use of an extracorporeal filter (e.g., CytoSorb®) as part of an ECMO treatment or cardiopulmonary bypass for any one of the subjects provided herein.

Such treating can include with an agent that inhibits or reduces cell free-DNA (cf-DNA). Such agents are referred to herein as inhibitors of cf-DNA. An “inhibitor of cf-DNA” is any agent that reduces or inhibits the amount of cf-DNA and/or its contribution to a pro-inflammatory response. Such agents include those that degrade cf-DNA. Other agents include those that block the production of cf-DNA. Still others are those that block the pro-inflammatory activities of cf-DNA.

Examples of cf-DNA inhibitors include, but are not limited to cationic nanoparticles (Liang et al., Nat Commun. 2018; 9: 4291) and deoxyribonucleases (DNases) (Cagliani et al., J of Surg Res., May 2020 (249): 104-113). DNases are enzymes that catalyze the hydrolytic cleavage of phosphodiester linkages in an DNA backbone. DNase I has been shown to increase survival of hemorrhaged mice having elevated levels of cf-DNA (Cagliani et al.). Examples of DNases include, but are not limited to, DNase I (e.g., recombinant human DNase I (rhDNase I) or bovine pancreatic DNase I), analogues of DNase I (such as, e.g., DNase X, DNase gamma, and DNAS1L2), DNase II (e.g., DNase II-alpha, DNase phosphodiesterase I, lactoferrin, and acetylcholinesterase. In one embodiment of any one of the methods provided herein, DNase I (e.g., PULMOZYME™) is administered. DNase I cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

Examples of cf-DNA inhibitors also include cationic polymers, which can neutralize cf-DNA. Other examples of cf-DNA inhibitors include inhibitors of the signaling pathway, including downstream, and blocking receptors for cf-DNA such as TLR9 or preventing TLR9 activation. Cell-free DNA is a stress signal in the danger associated molecular pattern (DAMP) pathway. Cf-DNA can activate Toll-like receptor 9 (TLR9) to secrete inflammatory cytokines. Blocking this TLR9 activation can potentially stop cytokine release/inflammatory response by stopping this immune response pathway. Thus, cf-DNA inhibitors include those that can block this activation (e.g., antibodies). Examples of downstream cf-DNA inhibitors include, but are not limited to, inhibitors of NLRP3 and IL-1. NLRP3 inhibitors include, but are not limited to, Cl— channel inhibitors (flufenamic acid, IAA94. DIDS, NPPB, etc.), G5, MCC950, JC124, colchicine, CY-09, ketone metabolite beta-hydroxubutyrate (BHB), a type I interferon, resveratrol, arglahin, CB2R, glyhenclamide, isoliquiritigenin, Z-VAD-FMK, and microRNA-223. IL-1 inhibitors include, but are not limited to, interleukin-1 receptor antagonists (e.g., IL-1ra) anti-IL-1 receptor monoclonal antibodies (e.g., canakinumab); IL-1 binding proteins (e.g., soluble IL-1 receptors (e.g., U.S. Pat. Nos. 5,492,888, 5,488,032, and 5,464,937, 5,319,071, and 5,180,812, the disclosures of which agents are hereby incorporated by reference herein)); anti-IL-1 monoclonal antibodies (e.g., anakinra, rilonacept); IL-1 receptor accessory proteins and antibodies thereto (e.g., WO 96/23067 and WO 99/37773, the disclosures of which agents are hereby incorporated by reference herein); inhibitors of interleukin-1 beta converting enzyme (ICE) or caspase I (e.g., N-benzyloxycarbonyl-Val-Ala-Asp-fluoromethylketone (z-VAD.FMK), acetyl-Tyr-Val-Ala-Asp-chloromethylketone, N-benzyloxycarbonyl-Asp-Glu-Val-Asp-fluoromethylketone), interleukin-1 beta protease inhibitors; and other compounds and proteins which block in vivo synthesis or extracellular release of IL-1.

In another embodiment, the therapy or treatment can comprise use of an inhibitor of IL-6 or the IL-6 receptor, such as human monoclonal antibodies against IL-6 receptor (e.g., tocilizumab (RoActemra, Roche) and sarilumab (Kevzara, Sanofi)).

As another example, cf-DNA inhibitors can include an agent that prevents release of cf-DNA through NETosis and/or blocks platelet formation or activation. Without being bound by any particular theory, apart from apoptotic and necrotic cell death, DNA release mechanisms include neutrophil extracellular trap release (NETosis). Platelet activation can trigger NETosis (which can increase cell-free DNA). Thus, agents that prevent platelet activation or formation and/or NETosis, can also be used as a cf-DNA inhibitor as provided herein. For example, such agents can include aspirin, heparin, etc.

In another embodiment, downstream decrease in cell-free DNA levels can be achieved by treatment the includes prone positioning and/or Remdesivir.

The cf-DNA inhibitors can be administered in effective amounts. “Amount effective” in the context of a composition for administration to a subject as provided herein refers to an amount of the composition or dosage form that produces one or more desired results in the subject, for example, the reduction or elimination of a pro-inflammatory immune response and/or a decrease in the level of cf-DNA in the subject, etc. The amount effective can be for in vitro or in vivo purposes. For in vivo purposes, the amount can be one that a clinician would believe may have a clinical benefit for a subject. In any one of the methods provided herein, the composition(s) administered may be in any one of the amounts effective as provided herein. In one embodiment of any one of the methods provided herein, the subject can be treated with a cf-DNA inhibitor when the amount of cf-DNA is above any one of the thresholds provided herein. In one embodiment of any one of the methods provided herein, the subject is treated with any one of the devices as provided herein including any one of the mechanical support devices provided herein that also includes a filter to reduce the level of cf-DNA in the subject.

The methods provided herein can include the administration of one or more additional therapies, treatments, etc.

In another embodiment, the additional treatment(s) is an ARDS treatment. ARDS treatments include, for example, prone positioning, sedation and medications to prevent movement, fluid management (e.g., diuretics), etc.

In another embodiment, the additional treatment(s) can be for pneumonia. Pneumonia treatments include, for example, cough suppressants, fever reducers (e.g., aspirin, acetaminophen), etc., although such treatments may also be applicable to any one of the subjects provided herein, and are also specifically contemplated for such subjects.

In another embodiment, the additional treatment(s) can be an anti-viral medication or any other medication known in the art. Other treatments include supportive care, such as rest and hydration.

In another embodiment, the additional treatment(s) can be a treatment for infection. In some embodiments, therapies for treating infection include therapies for treating a viral infection.

Other therapies are known to those of ordinary skill in the art. In some embodiments of any one of the methods provided herein, the treatment comprises one or more of the treatments described herein.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

The methods provided herein can include a step of assessing the subject. Alternatively, the methods provided herein can be performed on a subject assessed or identified as provided herein.

It has been found that total cell-free DNA (total cf-DNA) is correlated with tissue injury and can be used to assess and/or monitor a subject in a number of instances, such as in the transplant context or in post-operative cardiac surgery. The use of total cf-DNA can now be extended for assessing and monitoring a subject provided herein. Measuring circulating cf-DNA can rapidly and effectively assist the clinician in making assessments and can save lives while greatly unburdening the health care system. A subject so identified can be the subject of any one of the methods of treatment provided herein.

Cell-free DNA is found in very low concentrations in the plasma of healthy patients due to baseline cellular leakage during natural cell turnover. However, it can become very high when dying cells release DNA in much greater amounts than normal into the circulation. When optimized protocols for sample handling and qPCR analysis are used, the released cf-DNA can be measured very precisely, sensitively, quickly, and noninvasively across a wide range of concentrations as a biomarker of severity of illness using only a small peripheral blood sample that can be shipped at ambient temperature (e.g., 2 mls of blood collected by a simple peripheral blood draw). Such protocols have been tested and validated for the clinical-grade quantitative analysis of cf-DNA, as it has been applied clinically to cardiac surgical and transplant patients who are at heightened risk for not only cardiac, but multiorgan injury and failure. The utility of cf-DNA biomarker testing has been shown in over 7500 samples from 780 patients (540 cardiac transplant patients, 120 pediatric cardiac surgical patients, and 120 additional patients in two pilot studies). It has been found that cf-DNA levels begin to rise even before clinical symptoms become apparent, become higher with progression to clinically apparent illness, and are quantitatively correlated with longer term clinical outcomes. Cf-DNA levels over 50 ng/ml predicted increased likelihood of death, cardiac arrest, or mechanical circulatory support within 30 days (p=0.0001, AUC=0.89, NPV=0.99), levels over 25 ng/ml predicted longer hospital length of stay (greater than 30 days) (p≤0.01), and levels over 10 ng/ml predicted presence of infections (p<0.01) that go on to require clinical treatment.

As described herein, a proportional increase in cf-DNA level can be indicative of increasing severity and/or presence of one or more complications in the subjects described herein. Importantly, the short (15-30 min) half-life of individual cf-DNA molecules in the patient's plasma makes the cf-DNA concentration at any given time an accurate snapshot of the current level of risk in that patient at the time of sample collection. In one embodiment, any one of the methods of treatment provided herein can be of a subject identified with the methods of assessment as provided herein. In another embodiment, any one of the methods of treatment provided herein is of a subject determined to have a cf-DNA level as provided herein.

Early and accurate assessment can help the clinician get ahead of the condition or disease and can assist with decision making regarding need for intensive care measures including intubation. Additionally, cf-DNA levels would be expected to drop in response to successful therapy (in at least near real-time). Therefore, aspects of the disclosure relate, at least in part, to methods of quantifying total cf-DNA in a sample in order to assess or determine severity and/or complication or risk and/or response to therapy. In some embodiments, the subject on mechanical support and can be monitored with any one of the methods provided herein. Such subjects include surgical subjects, such as heart surgery subjects, in some embodiments.

As used herein, “cell-free DNA” (or “cf-DNA”) is DNA that is present outside of a cell, e.g., in the blood, plasma, serum, urine, saliva, etc. of a subject. “Total cell-free DNA” (or “total cf-DNA”) is the amount of cf-DNA present in a sample. Provided herein are methods and compositions that can be used to measure total cf-DNA, which may then be used to assess the subject's risk. The methods provided herein can be used to monitor a subject for worsening or improving condition. In any one of the methods provided herein, the method may further comprise performing one or more additional tests to assess the subject's condition.

A subject may be assessed by determining or obtaining one or more amounts of total cf-DNA. An amount of total cf-DNA may be determined with experimental techniques, such as those provided elsewhere herein or otherwise known in the art. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e., by purchasing), in some embodiments.

Because of the ability to determine amounts of cf-DNA, and the correlation with complications, risk, etc., the methods and compositions provided herein can be used to assess subjects. Thus, a risk of improving or worsening condition can be determined in such subjects. A “risk” as provided herein, refers to the presence or absence or progression of any undesirable condition in a subject, or an increased likelihood of the presence or absence or progression of such a condition. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or nonprogression) of such a condition.

As provided herein, early detection or monitoring of complications can facilitate treatment and improve clinical outcomes. Such methods can be used to monitor a subject over time. Further, such methods can aid in the selection, administration and/or monitoring of a treatment or therapy. Accordingly, the methods provided herein can be used to determine a treatment or monitoring regimen. The subject may be any one of the subjects provided herein. In one embodiment of any one of the methods provided herein, the subject is one that is on mechanical support or that is in need of mechanical support. such as ECMO or cardiopulmonary bypass.

The treatment and clinical course may be determined based on the subject's condition as determined as provided herein and/or the subject's associated expected outcome. For example, if the amount of total cf-DNA is 10 ng/mL or greater, 25 ng/mL or greater, or 50 ng/mL or greater, the subject may be treated with, or provided information related thereto, a therapy, such as those described above or elsewhere herein.

As used herein, “amount” refers to any quantitative value for the measurement of nucleic acids and can be given in an absolute or relative amount. Further, the amount can be a total amount, frequency, ratio, percentage, concentration, etc. As used herein, the term “level” can be used instead of “amount” but is intended to refer to the same types of values. Generally, unless otherwise provided, the amounts provided herein represent the total cf-DNA in a sample.

In some embodiments, any one of the methods provided herein can comprise comparing an amount to a threshold value, or to one or more prior amounts, to identify a subject at increased or decreased risk. In some embodiments of any one of the methods provided herein, a subject having an increased amount of total nucleic acids compared to a threshold value, or to one or more prior amounts, is identified as being at increased risk. In some embodiments of any one of the methods provided herein, a subject having a decreased or similar amount of total cf-DNA compared to a threshold value, or to one or more prior amounts, is identified as being at decreased or not increased risk.

“Threshold” or “threshold value” or “cutpoint”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold value of any one of the methods provided herein, can be any one of the threshold values provided herein, such as in the Examples or Figures.

The threshold values provided herein can be used to determine a risk of one or more complications or conditions in a subject. Accordingly, if the amount of total cf-DNA measured is equal to or greater than 8, 9, 10, 11, 12, 13, 14, 15, 16, 17, 18, 19, 20, 25, 30, 35, 40, 45, 50, 55, 60, 65, 70, 75 or 80 ng/mL, then the subject may be determined to be at increased risk of a complication or condition. For example, an amount equal to or greater than 50 ng/mL may be indicative of near-term severe clinical progression. The determination can be done based on any one of the comparisons as provided herein with or without other indicators of such a complication or condition.

The threshold values can also be used for comparisons to make treatment and/or monitoring decisions. For example, if the amount of total cf-DNA is greater than one of the thresholds provided herein and/or increasing over time, further monitoring may be indicated. As a further example, if the amount is greater than any one of the thresholds provided herein, treatment of the subject may be indicated. If the amount is greater than any one of the thresholds provided herein, additional testing of the subject, such as with a chest x-ray and/or computerized tomography (CT) may be indicated.

Accordingly, any one of the methods provided herein may further include an additional test(s) for assessing the subject, or a step of suggesting such further testing to the subject (or providing information about such further testing). The additional test(s) may be any one of the other methods provided herein or known in the art as appropriate. The type of additional test(s) will depend upon the condition of the subject and/or is well within the determination of the skilled artisan.

Exemplary additional tests for subjects provided herein, such as one suspected of ARDS include, but are not limited to, chest x-rays, computerized tomography (CT), lab tests, electrocardiograms, an echocardiograms.

Exemplary additional tests for subjects provided herein, such as one suspected of pneumonia include, but are not limited to, blood tests, chest x-rays, pulse oximetry, sputum tests, CT scans, and pleural fluid culture.

As another example, an additional test may be assessing the level of IL-6.

The amount of total cf-DNA may be determined by a number of methods. In some embodiments such a method is a sequencing-based method. Total cf-DNA may be analyzed using any suitable next generation or high-throughput sequencing technique.

In one embodiment, any one of the methods for determining total cf-DNA may be any one of the methods of U.S. Publication No. 2015-0086477-A1, and such methods are incorporated herein by reference in their entirety.

An amount of total cf-DNA may also be determined by a MOMA assay. In one embodiment, any one of the methods for determining total cf-DNA may be any one of the methods of PCT Publication No. WO 2016/176662 A1, and such methods are incorporated herein by reference in their entirety.

In some embodiments of any one of the methods provided herein, the method is an amplification-based quantitative assay, such as whereby nucleic acids are amplified and the amounts of the nucleic acids can be determined. Such assays include those whereby nucleic acids are amplified with the primers as described herein, or otherwise known in the art, and quantified. Such assays include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-stranded DNA-specific dye SYBR® Green (Molecular Probes). SYBR® Green is a dye that binds the minor groove of double-stranded DNA. When SYBR® Green dye binds to a double-stranded DNA, the fluorescence intensity increases. As more double-stranded amplicons are produced, SYBR® Green dye signal will increase.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I. T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or Sequenase. Exemplary polymerases include, but are not limited to Bacillus stearothermophilus pol I, Thermus aquaticus (Taq) pol I, Pyrccoccus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, MA), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, MO).

Salts and buffers include those familiar to those skilled in the art, including those comprising MgCl₂, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 50004 which may depend on the length and number of PCR products produced in a PCR reaction.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays.

Other methods for determining total cell-free DNA in a sample are known in the art. In some embodiments of any one of the methods provided herein, the total cell-free DNA is determined with TAQMAN™ Real-time PCR using RNase P as a target.

Any one of the methods provided herein can comprise extracting nucleic acids, such as total cell-free DNA, from a sample obtained from a subject. Such extraction can be done using any method known in the art or as otherwise provided herein (see, e.g., Current Protocols in Molecular Biology, latest edition, or the QlAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p.5205-5210; Circulating Nucleic Acids in Blood of Healthy Male and Female Donors. Clin. Chem. 2005, v. 51, p.1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu RWK, Poon LLM, Lau TK, Leung TN, Wong EMC, Lo YMD. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such an amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE head cleanup, head purification, or simply ExoSAP-IT™, or Zymo.

As used herein, the sample from a subject can be a biological sample. Examples of such biological samples include whole blood, plasma, serum, saliva, urine, etc. In some embodiments, addition of further nucleic acids, e.g., a standard, to the sample can be performed.

In another aspect, compositions and kits comprising one or more primer pairs as provided herein are provided. Other reagents for performing an assay, such as a PCR assay, may also be included in the composition or kit.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1— Total Cell-Free DNA (Cf-DNA) Test: Multi-Center Prospective Blinded Study

A multi-center prospective blinded study was undertaken to investigate the value of cf-DNA non-invasive clinical monitoring. 241 patients (aged 8 days to 73 years) were recruited from seven different sites. Of the patients, 146 were pediatric patients, and 95 were adults. The patients were longitudinally followed for at least one year. Samples were collected during routine catheterizations, hospital admissions, and events. In total, 2537 samples were analyzed in a blinded fashion. The relationship between total cf-DNA and death was analyzed. In all, 197 patients with 1150 samples were used for the analysis. There were 21 deaths over the study period. The mean cf-DNA values are shown below:

| | Death | Healthy | |
|---|---|---|---|
| cfDNA | 73.93 (5.17-777.08) ng/ml | 8.31 (0.07-1395.80) ng/ml | p = 0.004 |

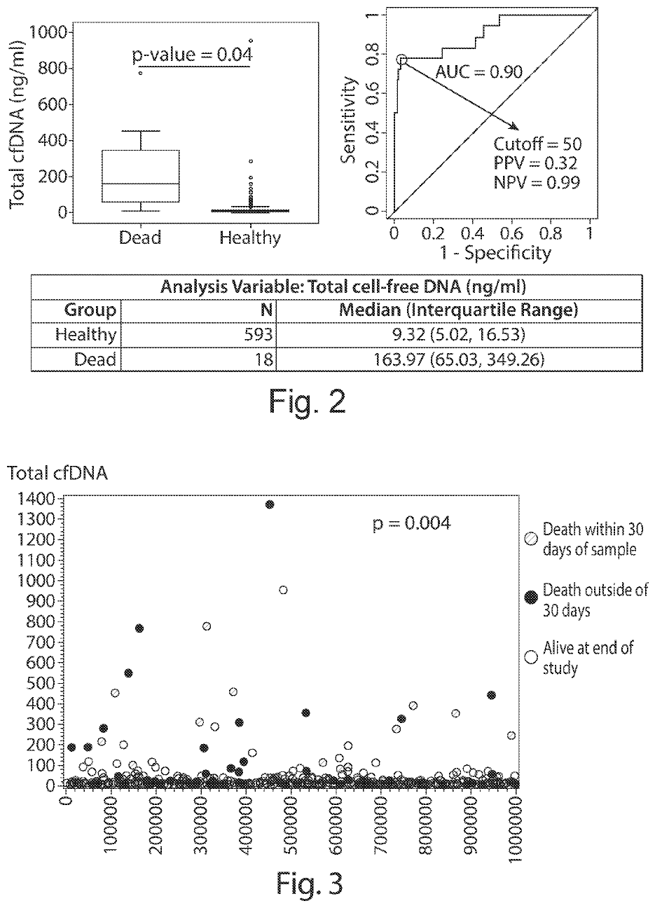

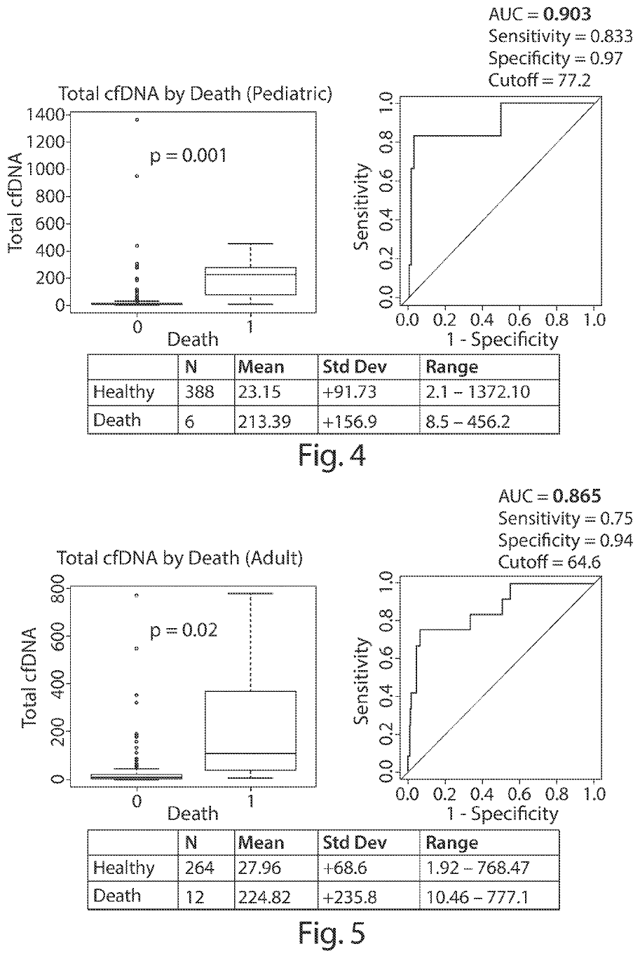

Graphs of the analysis using 50 ng/mL as the cutoff (threshold) are shown in FIG. 2. Total cf-DNA was found to predict clinical outcomes (death) as shown in FIG. 3. Whole blood and plasma samples were analyzed using ROC on repeated measures using correlation. The data was then examined for pediatric patients (FIG. 4) and adult patients (FIG. 5). The “healthy” group included samples not related to death (e.g., samples drawn more than 30 days before death) as well as those who did not die. Samples taken from patients within 7 days post-transplant were excluded from the analysis.

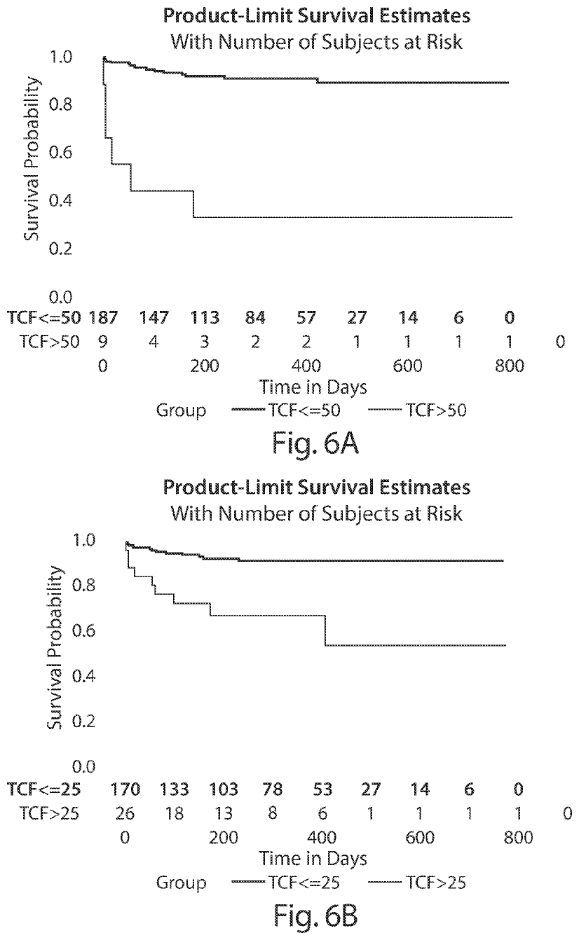

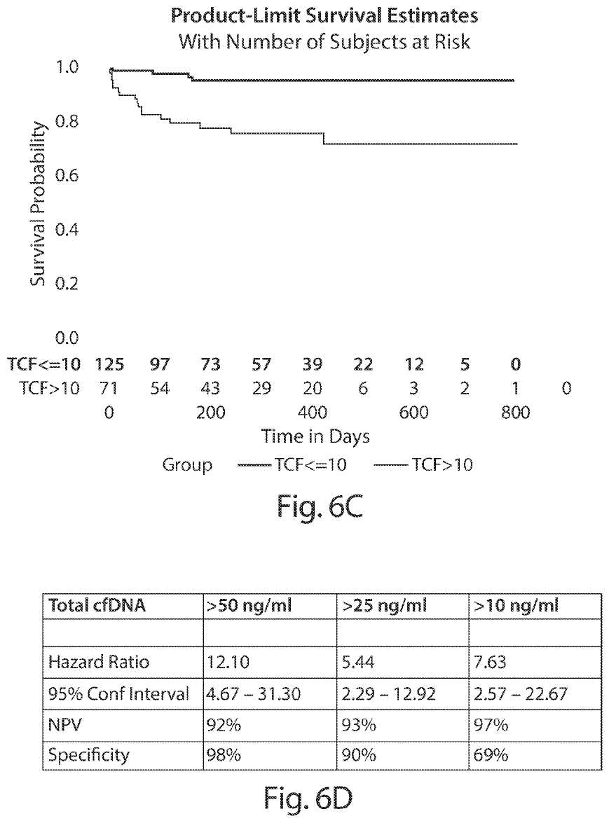

Cutoff values of 50 ng/ml, 25 ng/ml, and 10 n/ml were used to generate receiver operating characteristic (ROC) curves, which are shown in FIGS. 6A-6C. Data was graphed over time post-transplant. As can be seen in the table summarizing the results (FIG. 6D), the greatest specificity was observed with 50 ng/mL was used as the cut-off.

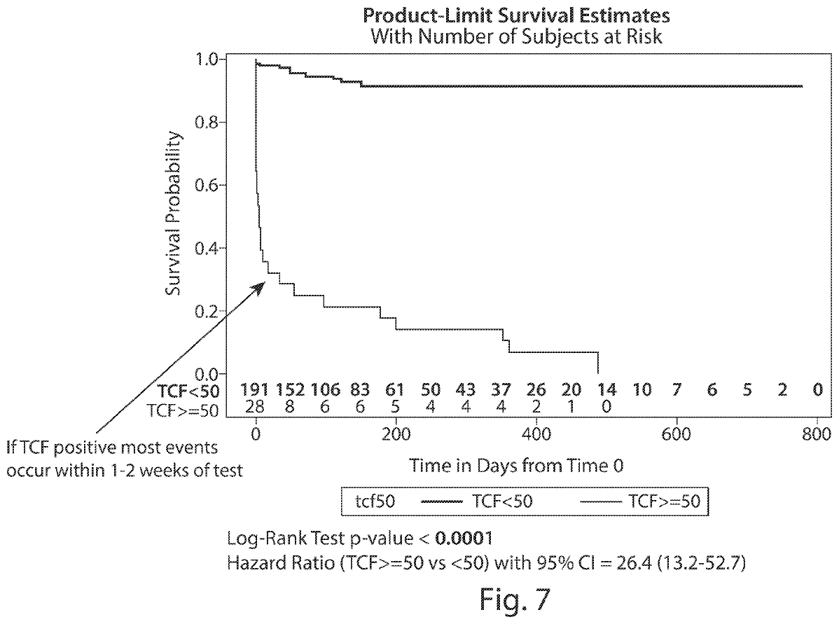

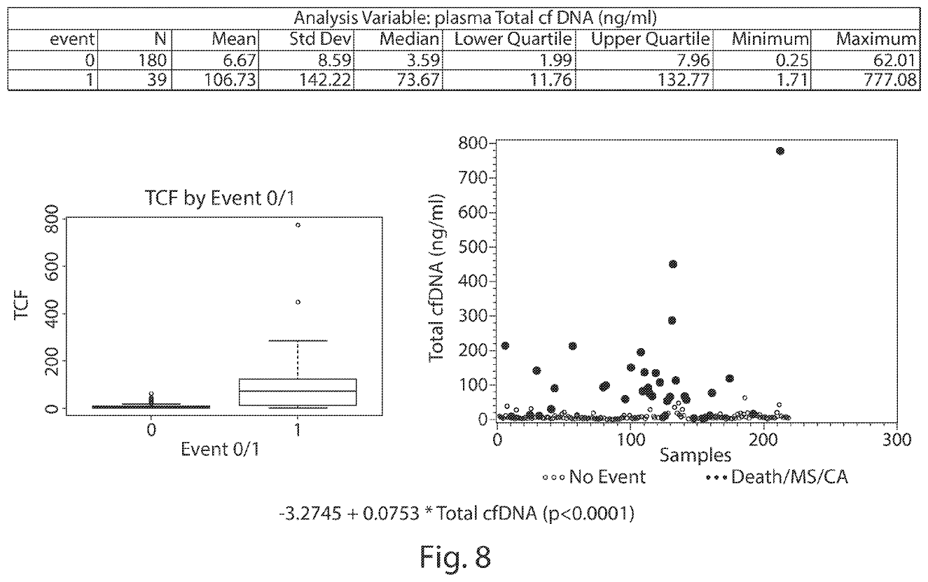

The data was analyzed for total cf-DNA and any event (death, cardiac arrest, or need for mechanical circulatory support). As can be seen in FIGS. 7-8, if the total cf-DNA is positive, most events occurred within 1-2 weeks of the test. The data is also presented in the table below (TCF=total cf-DNA):

| TCF50 | | | |
|---|---|---|---|
| Frequency | Event | | |
| Row Pct | | Death/MS/ | |
| Col Pct | No Event | Cardiac Arrest | Total |
| TCF <50 | 179 | 12 | 191 |
| | 93.72 | 6.28 | |
| | 99.44 | 30.77 | |
| TCF >=50 | 1 | 27 | 28 |
| | 3.57 | 96.43 | |
| | 0.56 | 69.23 | |
| Total | 180 | 39 | 219 |

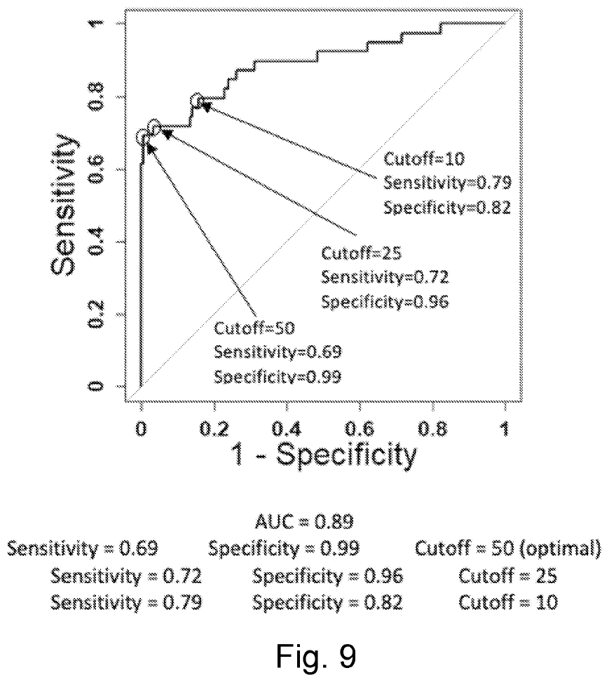

FIG. 9 shows an analysis of the different candidate cutoffs: 10 ng/ml, 25 ng/ml, and 50 ng/ml. As was demonstrated earlier, the 50 ng/ml cutoff provides the greatest specificity.

1. A method of treating a subject in need of mechanical support, the method comprising inhibiting the amount of cf-DNA in the subject.

2. The method of claim 1, wherein the subject is treated using mechanical support that comprises a filter that reduces the level of cf-DNA in the subject.

3. The method of claim 2, wherein the mechanical support comprises a ventilator, an ECMO device and/or a cardiopulmonary bypass device.

4. The method of claim 2, wherein the filter comprises CYTOSORB.

5. The method of claim 1, wherein the subject is treated by administration of a cf-DNA inhibitor to the subject.

6. The method of claim 1, wherein the method further comprises administering an additional therapy or treatment to the subject.

7. The method of claim 6, where the additional therapy or treatment is any one of the therapies or treatments provided herein.

8. The method of claim 1, wherein the subject is any one of the subjects described herein.

9. The method of claim 1, wherein the method further comprises determining an amount of cf-DNA in one or more samples from the subject.

10. The method of claim 1, wherein the method further comprises comparing the amount of cf-DNA to a threshold value or at least one prior amount of cf-DNA.

11. The method of claim 10, wherein the threshold is any one of the thresholds provided herein.

12. The method of claim 1, wherein the subject is one with, such as one who was determined to have, a level of cf-DNA greater than any one of the thresholds provided herein.

13. The method of claim 9, wherein the amount of cf-DNA is determined or obtained using an amplification-based quantification assay.

14. The method of claim 13, wherein the amplification-based quantification assay is quantitative real-time PCR (qRT-PCR) or digital PCR.

15. The method of claim 9, wherein the level of cf-DNA is determined in the subject daily.

16. The method of claim 1, wherein the mechanical support and/or filter are part of a system or circuit that is used on the subject.

17. A mechanical support system or circuit that comprises a mechanical support device and a filter that reduces cell-free DNA.

18. The mechanical support system or circuit of claim 17, wherein the mechanical support device comprises a ventilator, an ECMO device and/or a cardiopulmonary bypass device.

19. The mechanical support system or circuit of claim 17, wherein the filter comprises CYTOSORB.


SEQUENCE LISTING (2 sequences)

SEQ ID NO: 1 - protein, 4 aa, synthetic construct
  REGION 1..4: note = Synthetic
YVAD

SEQ ID NO: 2 - protein, 4 aa, synthetic construct
  REGION 1..4: note = Synthetic
DEVD